(19) United States
(12) Patent Application Publication
(10) Pub. No.: US 2002/0120319 A1
(43) Pub. Date: Aug. 29, 2002

(54) Housing structure of bioelectrode

(76) Inventors: Satoru Nishida, Kyoto (JP); Tsutomu Yamasawa, Kyoto (JP)

Correspondence Address: MORRISON & FOERSTER LLP, 2000 PENNSYLVANIA AVE, NW, SUITE 5500, WASHINGTON, DC, 20006-1888, US

(21) Appl. No.: 09/793,102

(22) Filed: Feb. 27, 2001

(51) Int. Cl. A61N 1/04

(52) U.S. Cl. 607/153

(57) ABSTRACT

The present invention provides a housing structure of a bioelectrode capable of maintaining moisture of the bioelectrode in equilibrium condition quickly without incurring decrease in adhesive strength due to over-dryness and damage. In a housing 10 for housing a bioelectrode which is used by coming into contact with an organism, the housing 10 including the bioelectrode-attaching portion 11 attaching a bioelectrode 1 is made of a water absorption material.

### TECHNICAL FIELD

The present invention relates to a housing structure for receiving a bioelectrode which is used by coming into contact with an organism in a low frequency therapeutic apparatus, an electrocardiograph and the like.

### BACKGROUND OF THE INVENTION

As a conventional housing of a bioelectrode, there is a thing that explained as below. For example, “A Conductive Element Housing” as described in Japanese Patent Laid-Open No. hei 10-248940 equips a housing plate which has a housing surface for receiving a conductive element (a bioelectrode) and an exothermic body for generating a heat of the housing plate, and it is configured to sterilize various germs stuck on the bioelectrode once the heat is applied as well as to restore an adhesive strength of the bioelectrode. Also, by installing an adhesive sheet of which the adhesive strength is weaker than that of the bioelectrode at the housing surface, dirt of the electrode is removed while detaching the bioelectrode from the housing surface.

On the other hand, in “An Electrically Conducting Adhesive Pad Storage Container” as described in Japanese Patent Laid-Open No. hei 7-31598, in order to maintain a relative humidity in a storage container which receives the electrically conducting adhesive pad (a bioelectrode), a humidity control agent is provided in the storage container and in cases that the bioelectrode is moisturized and absorbed by water used for removing sweat in use or dirt stuck on the bioelectrode, or the bioelectrode is dehumidified and dried by leaving it at a dry room for a long time, by storing the electrode in the storage container which the humidity is maintained constantly by the humidity control agent, the moisture of the electrode is maintained in a fixed equilibrium condition.

However, in the housing of the bioelectrode which equips said exothermic body, there are problems such as due to the heat of the exothermic body, the bioelectrode is excessively over-dried and deteriorates its adhesive strength, or when the bioelectrode is detached from the housing surface, the bioelectrode may be damaged due to adhesion of the adhesive sheet. Moreover, the storage container, which installs said humidity control agent has a problem that it takes considerable time to maintain the humidity of the bioelectrode which is moisturized and absorbed or dehumidified and dried in equilibrium condition.

### SUMMARY OF THE INVENTION

Considering above conventional problems, the object of the present invention is to provide a housing structure of a bioelectrode, which is capable of maintaining the moisture of the bioelectrode in equilibrium condition quickly, without deterioration of the adhesive strength due to over-dryness and concern of damage.

To accomplish said object, according to the housing structure of a bioelectrode as described in claim 1, it is characterized that the whole housing structure including a bioelectrode-attaching portion is made of a water absorption material. In this housing structure, since the housing structure itself is made of the water absorption material, once the bioelectrode is received, i.e., the bioelectrode is attached to the bioelectrode-attaching portion, the moisture of the bioelectrode is absorbed by the water absorption material.

According to the housing structure of a bioelectrode as described in claim 4, it is characterized that only a bioelectrode-attaching portion has a water absorption means. In this housing structure, since the bioelectrode-attaching portion has the water absorption means (e.g., a water absorption material), similarly, once the bioelectrode is attached to the bioelectrode-attaching portion, the moisture of the bioelectrode is absorbed by the water absorption means.

According to the housing structure of a bioelectrode as described in claim 8, it is characterized that a portion other than a bioelectrode-attaching portion has a water absorption means. In this housing structure, the portion other than the bioelectrode-attaching portion has the water absorption means (e.g., a water absorption material), for example, in the first place, the bioelectrode is attached to the water absorption means and the moisture of the bioelectrode is absorbed into the water absorption means and then the bioelectrode is attached and can be received to the bioelectrode-attaching portion.

According to the housing structure of a bioelectrode as described in claim 12, it is characterized that a bioelectrode-attaching portion has a surface of convex-concave shape. In this housing structure, since the surface of the bioelectrode-attaching portion is the convex-concave shape, once the bioelectrode is attached to the bioelectrode-attaching portion, a gap between the bioelectrode and the surface of the bioelectrode-attaching portion is formed and through this gap, the moisture of the bioelectrode is evaporated.

According to the housing structure of a bioelectrode as described in claim 14, it is characterized that a bioelectrode-attaching portion has a plurality of through-holes, perforated through corresponding portions thereof from front surface to rear surface. In this housing structure, once the bioelectrode is attached to the bioelectrode-attaching portion, the moisture of the bioelectrode is evaporated through the through-holes. In the housing structures as described in claims 1, 4 and 8, when there is excessive moisture in the bioelectrode, the moisture is absorbed by the water absorption material acting as the water absorption means, and conversely, when moisture necessary for the bioelectrode is insufficient, the moisture of the water absorption material is absorbed into the bioelectrode. That is, because the water absorption material functions as a humidity control agent which adjusts the humidity in accordance with a moisture content or a dryness of the bioelectrode, the moisture of the bioelectrode is maintained in equilibrium condition quickly, and there is no case that the bioelectrode is excessively over absorbed or excessively over dried. Further, since the bioelectrode is attached to the water absorption material itself or to the bioelectrode-attaching portion simply (because it is not attached on an adhesive sheet), it is free from damage.

Also, according to the housing structures as described in claims 12 and 14, once the bioelectrode is attached to the bioelectrode-attaching portion, an airflow passage is formed, thus, the bioelectrode is moisturized or dehumidified through the airflow passage, thereby the moisture of the bioelectrode is maintained in equilibrium condition. In addition, because the bioelectrode is attached to the bioelectrode-attaching portion simply (because it is not attached on an adhesive sheet), it is free from damage.

Furthermore, in accordance with the present invention, the water absorption material as the water absorption means is not specified if it is excellence in absorptive property and unharmed to the human body. The water absorption material may be made of polyurethane, vinyl chloride, polyethylene, silicon rubber, EPDM rubber and etc and may be a non-woven fabric, cloth and a foamed body with continuous foams.

### BRIEF DESCRIPTION OF THE DRAWINGS

The embodiments of the present invention will be explained with reference to the accompanying drawings, in which:

FIG. 1 is a perspective view of a housing having a housing structure installed therein in accordance with a preferred embodiment;

FIG. 2 is a cross sectional view of the housing shown in FIG. 1;

FIG. 3 is a perspective view of a housing having a housing structure installed therein in accordance with another preferred embodiment;

FIG. 4 is a cross sectional view of the housing shown in FIG. 3;

FIG. 5 is a perspective view of housing having a housing structure installed therein in accordance with another preferred embodiment;

FIG. 6 is a cross sectional view of the housing shown in FIG. 5;

FIG. 7 is a perspective view of an electronic equipment body having a housing structure installed therein in accordance with another preferred embodiment;

FIG. 8 is a cross sectional view of the electronic equipment body shown in FIG. 7;

FIG. 9 is a perspective view of a housing having a housing structure installed therein in accordance with another preferred embodiment;

FIG. 10 is a cross sectional view of the housing shown in FIG. 9;

FIG. 11 is a perspective view of a housing having a housing structure installed therein in accordance with another preferred embodiment;

FIG. 12 is a cross sectional view of the housing shown in FIG. 11;

FIG. 13 is a perspective view of a housing having a housing structure installed therein in accordance with another preferred embodiment;

FIG. 14 is a cross sectional view of the housing shown in FIG. 13;

FIG. 15 is a perspective view of a housing having a housing structure installed therein in accordance with another preferred embodiment;

FIG. 16 is a cross sectional view of the housing shown in FIG. 15;

FIG. 17 is a perspective view of a housing having a housing structure installed therein in accordance with another preferred embodiment;

FIG. 18 is a cross sectional view of the housing shown in FIG. 17;

FIG. 19 is a perspective view of a housing having a housing structure installed therein in accordance with another preferred embodiment;

FIG. 20 is a cross sectional view of the housing shown in FIG. 19;

FIG. 21 is a perspective view of a housing having a housing structure installed therein in accordance with another preferred embodiment and

FIG. 22 is a cross sectional view of the housing shown in FIG. 21.

### DETAILED DESCRIPTION OF THE INVENTION

In below, the present invention is described based on the embodiment. However, the present invention has a characteristic in a housing structure of a bioelectrode, and since the configuration of the bioelectrode other than the housing structure or an electronic equipment body may be similar to the prior art and therefore a description is focused on the housing structure.

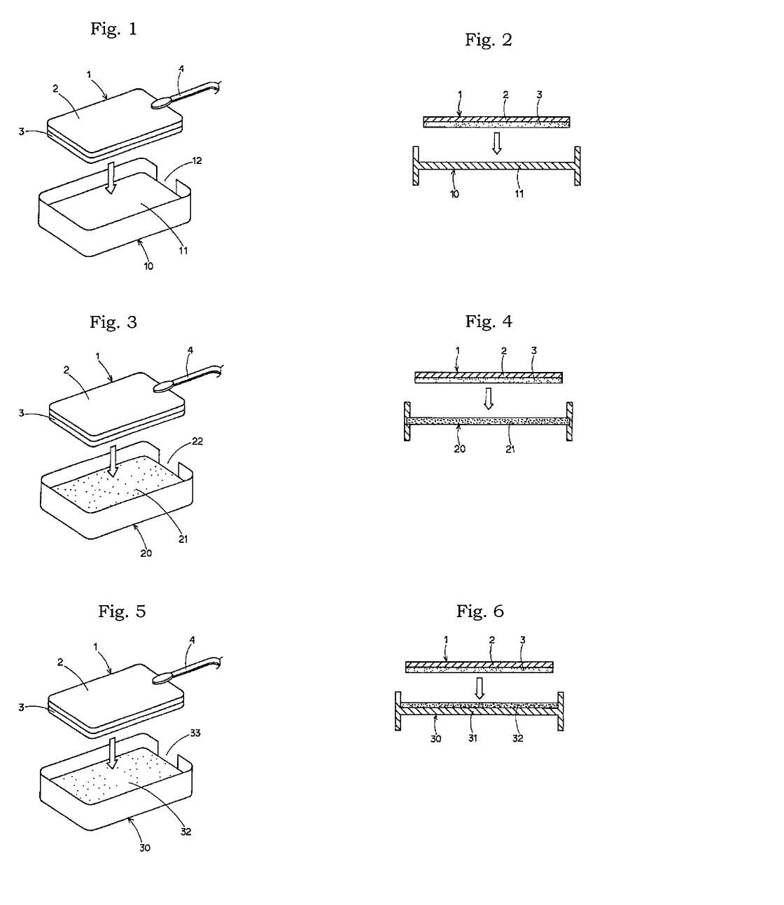

A housing structure of a bioelectrode in accordance with a preferred embodiment is shown in a perspective view, FIG. 1 and a cross sectional view, FIG. 2. This housing structure is installed at a housing which is separately prepared from an electronic equipment body such as a low frequency therapeutic apparatus, an electrocardiograph and the like, wherein a bioelectrode 1 is attached and received to a housing 10. The bioelectrode 1 has an electrode body 2, an electrically conductive adhesive pad 3 adhered to the electrode body 2, and a cord 4 connecting between the electrode body 2 and an electronic equipment body (not shown).

A housing 10 has a bioelectrode-attaching portion 11 to which the bioelectrode 1 is attached and a notch portion 12 for passing the cord 4 while attaching the bioelectrode 1 to the bioelectrode-attaching portion 11. The whole housing 10 including the bioelectrode-attaching portion 11 is made of the water absorption material mentioned above. Once the bioelectrode 1 is received in this housing 10, i.e., once the bioelectrode 1 is attached to the bioelectrode attaching portion 11, the residual moisture of the bioelectrode 1 is absorbed by the water absorption material, which forms the whole housing 10. That is, when there is excessive moisture in the bioelectrode 1, the residual moisture is absorbed by the water absorption material, and conversely, when there is insufficient moisture in the bioelectrode 1, the moisture of the water absorption material is supplemented to the bioelectrode 1. Accordingly, the water absorption material functions as a humidity control agent, which adjusts the humidity in accordance with a moisture content or a dryness of the bioelectrode 1, the moisture of the bioelectrode 1 is maintained in equilibrium condition quickly, and prevents the bioelectrode 1 from moisturizing or drying excessively. Further, since the bioelectrode 1 is attached to the bioelectrode-attaching portion 11 made of the water absorption material, the bioelectrode 1 is free from damage in comparison with the case that it is attached to the adhesive sheet.

A housing structure in accordance with another preferred embodiment is shown in a perspective view, FIG. 3 and a cross sectional view, FIG. 4. Similarly, this housing structure is installed at a housing 20 and the housing 20 has a bioelectrode-attaching portion 21 and a notch portion 22. In this housing 20, only the bioelectrode-attaching portion 21 is made of the water absorption material. Once the bioelectrode 1 is attached to the bioelectrode-attaching portion 21 in this housing 20, the moisture of the bioelectrode 1 is maintained in equilibrium condition by the attaching portion of a water absorptive property 21. The bioelectrode 1 is free from damage since it is attached to the bioelectrode-attaching portion 21 made of the water absorption material.

A housing structure in accordance with another preferred embodiment is shown in a perspective view, FIG. 5 and a cross sectional view, FIG. 6. Similarly, this housing structure is installed at a housing 30. In contrast to said housing 20, in this housing 30, a water absorption material 32 is installed separately as a supplementary material at a bioelectrode-attaching portion 31. In this case, comparing with the housing 20, a water absorption material 32 is installed at the bioelectrode-attaching portion 31 after forming the whole housing 30 including the bioelectrode-attaching portion 31, thereby making it possible to decrease a production cost. Further, this housing 30 obtains an equal functional effect as that of said housing 20.

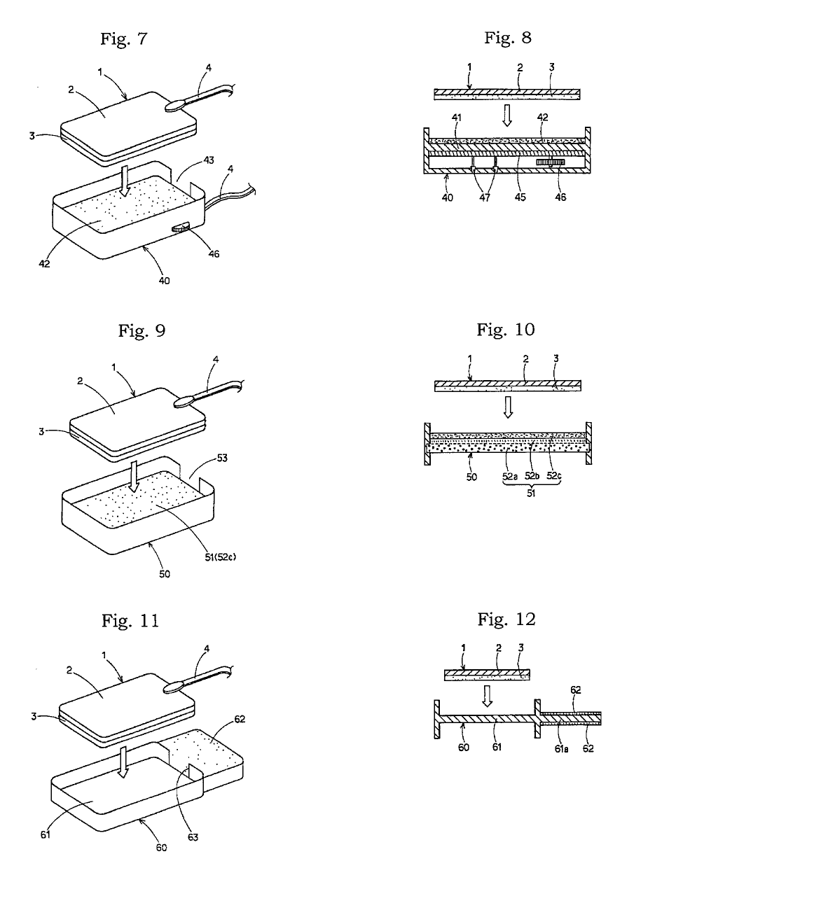

This housing 30 is different from the electronic equipment body; however, an example that the housing structure is installed at the electronic equipment body is shown in FIG. 7 (a perspective side view) and FIG. 8 (a cross sectional view). Here, the bioelectrode 1 is received in receiving portion allocated at rear surface of an electronic equipment body 40 and a water absorption material 42 is installed at a bioelectrode-attaching portion 41 of the electronic equipment body 40. Also, at the attaching side and the opposite side of the bioelectrode 1 of a bioelectrode-attaching portion 41, a circuit board 45 is allocated and the circuit board 45 has, for example, a control dial 46 to operate a power supply ON/OFF and a LED 47 serving to display an operation confirmation indication thereon. The control dial 46 is installed at the side of the electronic equipment body 40 to operate manually and the LED 47 is exposed at the surface of the electronic equipment body 40.

On the other hand, in the housing 30 and the electronic equipment body 40, while the water absorption materials 32 and 42 are formed in a layer respectively but it does not matter if they are formed in a layered structure of two layers or more. In this case, for example, in a two-layered structure, a combination of an absorption layer and an evaporation layer or that of the absorption layer and a water accumulation layer may be shown and in a three-layered structure, a combination of the absorption layer, the water accumulation layer and the evaporation layer may be shown. The absorption layer serves to absorb moisture, the evaporation layer serves to evaporate the moisture and the water accumulation layer serves to store proper moisture in advance. An adequate combination of these layers prevents the excessive moisture or over dryness, thereby it is possible to maintain the moisture of the bioelectrode quickly in equilibrium condition.

An example of a housing structure, which equips a water absorption material of three-layer structure, is shown in FIG. 9 (a perspective view) and FIG. 10 (a cross sectional view). In a housing 50, having this housing structure installed therein, a bioelectrode-attaching portion 51 is formed of the three-layered structure of water absorption materials 52a, 52b and 52c. The water absorption materials 52a, 52b and 52c are evaporation layers, a water accumulation layer and a water absorption layer respectively in order and the bioelectrode 1 is directly attached to the water absorption layer 52c.

Any of said housings 10, 20, 30 and 50, or the electronic equipment body 40 attaches the bioelectrode 1 to the water absorption material, however, an example that the water absorption material may be used at a portion other than the bioelectrode-attaching portion is shown in FIG. 11 (a perspective view) and FIG. 12 (a cross sectional view). In here, a bioelectrode-attaching portion 61 of a housing 60 is extended and a water absorption material 62 is installed on an extended portion 61a (a portion other than the bioelectrode-attaching portion).

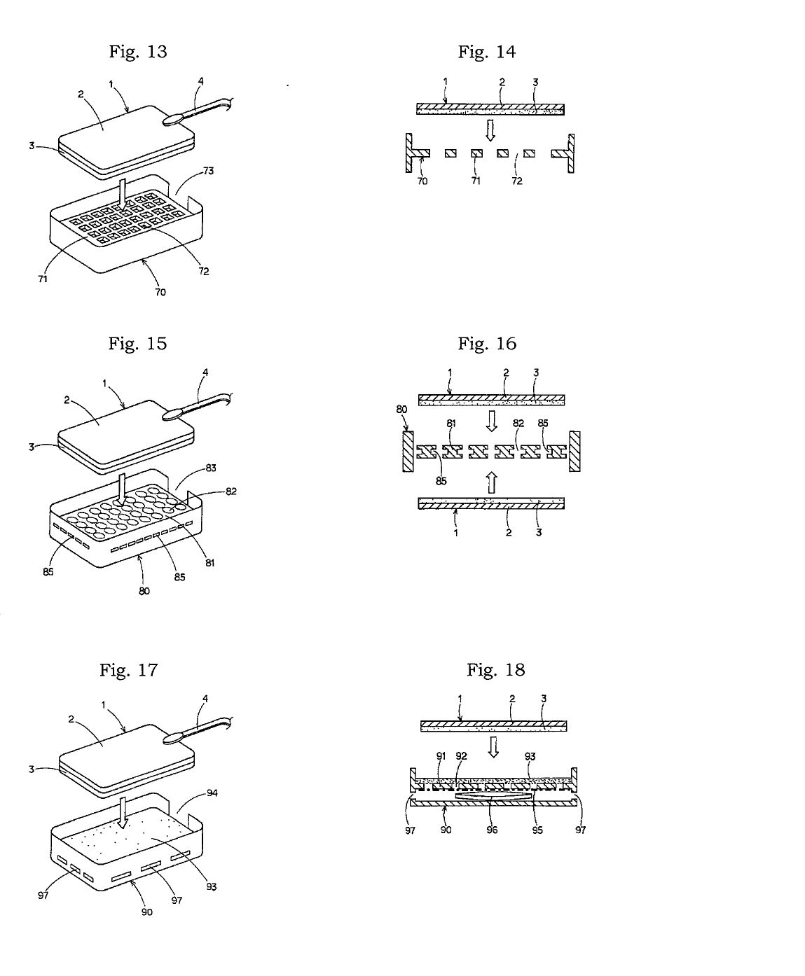

In this housing 60, the bioelectrode 1, after being rinsed by water, is attached to the water absorption material 62 first and absorbs the moisture of the bioelectrode 1 to the water absorption material 62 and is configured to attach and store the bioelectrode 1 to the bioelectrode-attaching portion 61 for the storage thereof. Any of said housings 10, 20, 30, 50 and 60, and the electronic equipment body 40 use the water absorption material, however, an example which does not use the water absorption material is shown in FIG. 13 (a perspective view) and FIG. 14 (a cross sectional view). The bioelectrode-attaching portion 71 of the housing 70 has a plurality of through-holes 72 perforated through corresponding portions thereof from front surface to rear surface. The through-holes 72 with rectangular cross-section shape are arranged in the form of a grid. But, the through-holes 72 may be arranged irregularly and the size and shape thereof may be varied.

In this housing 70, once the bioelectrode 1 is attached to the bioelectrode-attaching portion 71, a portion of the bioelectrode 1, which faces with the through-holes 72, is out of contact with the bioelectrode-attaching portion 71. Thus, the moisture of the bioelectrode 1 is evaporated through the through-holes 72 or conversely, the moisture is absorbed into the bioelectrode 1 thereby, the moisture of the bioelectrode 1 is maintained in equilibrium condition. Further, since the bioelectrode 1 is attached to the bioelectrode-attaching portion 71 having the through-holes 72, it is free from damage.

A housing structure in accordance with another preferred embodiment is shown in FIG. 15 (a perspective view) and FIG. 16 (a cross sectional view). In the housing 80, to attach the bioelectrode 1 to both sides for a bioelectrode-attaching portion 81, the bioelectrode-attaching portion 81 has a through-hole 82, and at the same time, grooves 85 which perpendicularly communicate with the through-holes 82 in a front and rear direction of the through-holes 82. The through-holes 82 with circular cross-section shape are arranged in a grid form, and the grooves 85 pass horizontally through the bioelectrode-attaching portion 81 and allow all of the through-holes 82 to communicate with each other in horizontal and vertical directions. Also, the through-holes 82 are opened at the side of the housing 80 and communicate with the outside.

In the housing 80, when the bioelectrode 1 is attached to the both sides of the bioelectrode-attaching portion 81, the through-holes 82 are obstructed with the bioelectrode 1, but all the through-holes 82 communicate with each other by the grooves 85 which communicate with the outside of the housing 80, thereby permitting the moisture of the bioelectrode 1 to be evaporated to the outside of the housing 80 through the grooves 85 or allowing the outside moisture to be absorbed into the bioelectrode 1 through the grooves 85, thus, benefits similar to that of the housing 70 are obtained.

A housing structure in accordance with another preferred embodiment is shown in FIG. 17 (a perspective view) and FIG. 18 (a cross sectional view). A housing 90 is provided with a water absorption material 93 disposed on a bioelectrode-attaching portion 91 having through-holes 92, and a heater 95 (e.g., PTC heater) and a fan 96 mounted at opposite side to the water absorption material 93, each of which evaporate moisture. The heater 95 is mounted on the surface of the bioelectrode-attaching portion 91 in a predetermined wiring pattern, and the fan 96 is mounted to blow air through the through-holes 92 toward the water absorption material 93. Further, openings 97 are formed on the side of the housing 90, and the inside of the housing 90 communicates with the outside through the openings 97. While both the heater 95 and the fan 96 are provided in the present invention, any one of them may be employed.

In the housing 90, after the bioelectrode 1 is attached to the water absorption material 93, for example, if a switch (not shown) that is separately mounted in the housing 90 is turned ON to apply an electric current to the heater 95, evaporation of moisture absorbed into the water absorption material 93 is accelerated by heat from the heater 95, thereby an absorptive property of the water absorption material 93 is raised. In addition, the operation of the fan 96 allows air to flow toward the water absorption material 93, and permits the water vapor to be exhausted from the openings 97 to the outside of the housing 90, thereby the evaporation of the moisture is further accelerated.

Further, in the housings 70, 80 and 90, any of the bioelectrode-attaching portions with the through-holes can be formed by means of a mold process, for example. And, the bioelectrode-attaching portion 81 of the housing 80 may be constituted by a combination of a plurality of plates. For example, two plates with small through-holes and one plate with through-holes larger than those of the two plates may be used, and the one plate with the large through-holes may sandwiched between the two plates with the small through-holes, thereby the through-holes may communicate with each other.

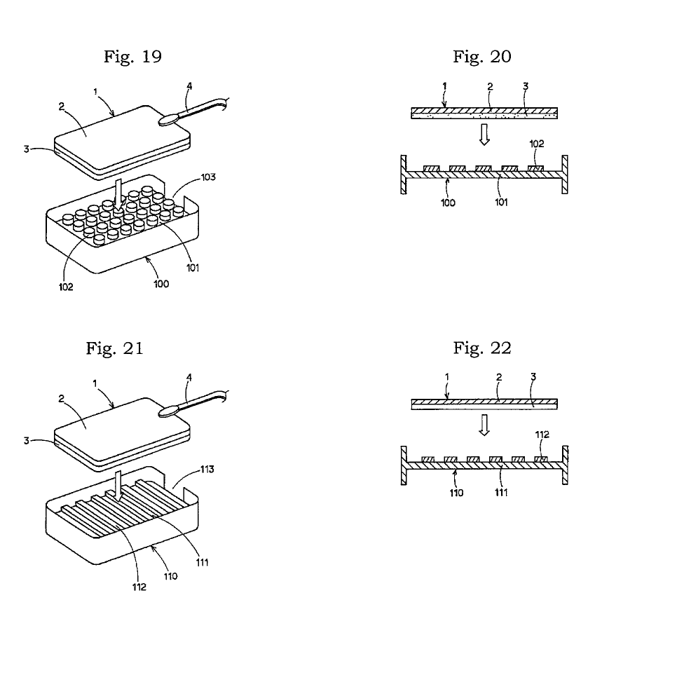

A housing structure in accordance with another preferred embodiment is shown in FIG. 19 (a perspective view) and FIG. 20 (a cross sectional view). In a housing 100 of this embodiment, the surface of a bioelectrode-attaching portion 101 has a convex-concave shape wherein columnar convex members 102 is formed side by side on the bioelectrode-attaching portion 101 lengthwise and crosswise. The formation of the columnar convex members 102 on the surface of the bioelectrode-attaching portion 101 forms concave members around the columnar convex members 102. Accordingly, in the housing 100, when the bioelectrode 1 is attached to the bioelectrode-attaching portion 101, the concave members function as an airflow passage, and thus, benefits similar to the case of the bioelectrode-attaching portions having the through-holes are obtained.

Further, for example, the concave members communicate with the outside of the housing 100 by forming an opening in the side of the housing 100 or forming a notch portion therein, and the inside of the housing 100 communicates with the outside, thereby moisture and water can be easily drained to the outside of the housing 100 in more preferred from. Another example of the convex members is shown in FIG. 21 (a perspective view) and FIG. 22 (a cross sectional view). In a housing 110, prismatic convex members 112 formed on the surface of a bioelectrode-attaching portion 111. The housing 110 has concave members formed by the convex members 112, and thus, benefits similar to that of the housing 100 are obtained.

As another shape of the columnar convex members 102 and prismatic convex members 112, the surface of the bioelectrode-attaching portion may be formed in a wave shape. If the surface of the bioelectrode-attaching portion has a convex-concave shape, a shape of convex member and the number thereof are not limited. Further, said convex members 102 and 112 may be separately or integrally provided in the surface of the bioelectrode-attaching portion, or the surface of the convex-concave shape may be formed by means of a crimp process based on a mold.

Further, the water absorption material of multi-layered structure shown in the housing 50 may be applied to another embodiment other than the foregoing embodiments.

As explained in the above, according to any of the housing structures as described in claims 1-11 of the present invention, when a considerable amount of moisture is included in a bioelectrode, a water absorption material acting as a water absorbing means absorbs the moisture, and to the contrary, when moisture necessary for the bioelectrode is insufficient, the moisture of the water absorption material is absorbed into the bioelectrode. In short, since the water absorption material functions as a humidity control agent which adjusts humidity in accordance with a moisture content or a dryness of the bioelectrode, the moisture of the bioelectrode is maintained in equilibrium condition quickly, thereby preventing the bioelectrode from being excessively absorbed or being excessively dried. Furthermore, since the bioelectrode is attached to the water absorption material itself or the bioelectrode-attaching portion simply (because it is not attached on an adhesive sheet), it is free from damage.

Moreover, according to any of the housing structures as described in claims 12-17 of the present invention, when the bioelectrode is attached to the bioelectrode-attaching portion, an airflow passage is formed, thus the bioelectrode is moisturized or dehumidified through the airflow passage, thereby the moisture of the bioelectrode is maintained in equilibrium condition. In addition, since the bioelectrode is attached to the bioelectrode-attaching portion simply (because it is not attached on an adhesive sheet), it is free from damage.

Furthermore, the present invention employs a heater or a fan to elevate an absorptive property by the water absorption material, which absorbs moisture of the bioelectrode or an evaporative property through the airflow passage, and also employs both the heater and the fan to further increase the advantages previously described.

What is claimed is:

1. A housing structure of a bioelectrode with a bioelectrode-attaching portion for receiving a bioelectrode which is used by coming into contact with an organism, wherein the whole housing structure including said bioelectrode-attaching portion is made of a water absorption material.

2. A housing structure of a bioelectrode according to claim 1, wherein said water absorption material is a foamed body.

3. A housing structure of a bioelectrode according to claim 1 or 2, wherein said water absorption material has a layered structure of two layers or more.

4. A housing structure of a bioelectrode with a bioelectrode-attaching portion for receiving a bioelectrode which is used by coming into contact with an organism, wherein only the bioelectrode-attaching portion includes a water absorption means.

5. A housing structure of a bioelectrode according to claim 4, wherein said water absorption means is made of a water absorption material.

6. A housing structure of a bioelectrode according to claim 4 or 5, wherein said water absorption means is a foamed body.

7. A housing structure of a bioelectrode according to any one of claims 4 to 6, wherein said water absorption means has a layered structure of two layers or more.

8. A housing structure of a bioelectrode with a bioelectrode-attaching portion for receiving a bioelectrode which is used by coming into contact with an organism, wherein a portion other than said bioelectrode-attaching portion includes a water absorption means.

9. A housing structure of a bioelectrode according to claim 8, wherein said water absorption means is made of a water absorption material.

10. A housing structure of a bioelectrode according to claim 8 or 9, wherein said water absorption means is a foamed body.

11. A housing structure of a bioelectrode according to any one of claims 8 to 10, wherein said water absorption means has a layered structure of two layers or more.

12. A housing structure of a bioelectrode with a bioelectrode-attaching portion for receiving a bioelectrode which is used by coming into contact with an organism, wherein said bioelectrode-attaching portion has a surface of convex-concave shape.

13. A housing structure of a bioelectrode according to claim 12, wherein concave members on the surface of the convex-concave shape communicate with the outside of the housing structure.

14. A housing structure of a bioelectrode with a bioelectrode-attaching portion for receiving a bioelectrode which is used by coming into contact with an organism, wherein said bioelectrode-attaching portion includes a plurality of through-holes perforated through corresponding portions thereof from front surface to rear surface.

15. A housing structure of a bioelectrode according to claim 14, wherein said through-holes are arranged in the form of a grid.

16. A housing structure of a bioelectrode according to claim 14, wherein said through-holes are arranged irregularly.

17. A housing structure of a bioelectrode according to any one of claims 14 to 16, wherein said bioelectrode-attaching portion includes grooves, which communicate with the through-holes perpendicularly to thickness direction.

18. A housing structure of a bioelectrode according to any one of claims 1 to 17, wherein a heater is mounted for evaporating the moisture toward the opposite side to the bioelectrode attaching side of said bioelectrode-attaching portion.

19. A housing structure of a bioelectrode according to any one of claims 1 to 17, wherein a fan is mounted for evaporating the moisture toward the opposite side to the bioelectrode attaching side of said bioelectrode-attaching portion.

20. A housing structure of a bioelectrode according to any one of claims 1 to 17, wherein a heater and a fan are mounted for evaporating the moisture toward the opposite side to the bioelectrode attaching side of said bioelectrode-attaching portion.

21. A housing structure of a bioelectrode according to any one of claims 1 to 20, wherein said housing structure is mounted to a housing, which is separately prepared from the electronic equipment body.

22. A housing structure of a bioelectrode according to any one of claims 1 to 20, wherein said housing structure is mounted to the electronic equipment body.
